(19) United States
(12) Patent Application Publication
(10) Pub. No.: US 2012/0308538 A1
(43) Pub. Date: Dec. 6, 2012

(54) PREVENTING SPOILAGE IN ALCOHOL FERMENTATIONS

(76) Inventors: Ursula Bond, Dublin (IE); Tharappel C. James, Skerries (IE); Joanne Kavanagh, Dublin (IE)

(21) Appl. No.: 13/482,223

(22) Filed: May 29, 2012

Related U.S. Application Data
(60) Provisional application No. 61/491,584, filed on May 31, 2011.

(51) Int. Cl. C12C 11/00; C12G 1/00; A61P 31/04; C12P 7/06; C07C 31/08; A61K 38/17; C12G 3/00; C12N 1/19

(52) U.S. Cl. 424/93.21; 426/11; 426/592; 426/15; 426/16; 435/254.2; 435/254.21; 435/161; 568/840

(57) ABSTRACT

The invention relates to the prevention of bacterial spoilage during fermentation. In particular it relates to the prevention of beer and wine spoilage, but also the spoilage of bioethanol or biogas. This is accomplished by modifying the fermentation yeast to produce an antimicrobial peptide known as a defensin. One such defensin is the human β-defensin-3 (HBD-3) although many others can be used.

### FIELD OF THE INVENTION

The invention relates to the prevention of bacterial spoilage during fermentation. In particular it relates to the prevention of beer and wine spoilage, but also the spoilage of other alcohols, and bioethanol or biogas. This is accomplished by modifying the fermentation yeast to produce an antimicrobial peptide known as a defensin. One such defensin is the human β-defensin-3 (HBD-3) although many others can be used.

### BACKGROUND TO THE INVENTION

Conventional brewing has over 8000 years of history. It can be regarded as the first example of traditional biotechnology. Beer is one of the most popular alcoholic beverages over the years. The brewing industry is a huge global business and this dynamic sector is open to new developments in technology and scientific progress. Brewer's are also very concerned that any new techniques they adopt are the best in terms of product quality, cost effectiveness and brand maintenance.

During conventional beer production, the major sugar source, barley grain, goes through physiochemical processes (mashing, boiling) to produce the wort. Hops (Humulus lupulus) may be added during boiling to provide bitterness and protect against bacterial spoilage. The insoluble residue produced during the process is of economic importance, for example, both the wort separation and clarification residues are used in biofeed for animal stock. This yeast biomass is also a rich source of macro and micronutrients for all forms of life including humans.

Modern day fermenters are very large cylindro-conical containers of 5-10,000 hectoliters and their sterility is of most importance to the brewing industry. The sole living organism in the fermenter is the yeast. Brewery yeasts are allotetrapolyploid hybrid strains of the genus Saccharomyces pastorianus, previously known as Saccharomyces carlsbergenis. Fermentations are carried out at low temperatures (8-15° C.) and generally contain ten million yeast cells. At the end of fermentation, the yeasts form a cloudy mass and fall (flocculate) to the bottom of the vessel. The yeasts are thus referred to as bottom fermenters. Lager beers produced by bottom-fermenting yeasts are the most widespread beer types throughout the world (more than 90%). To produce ale beers, strains of Saccharomyces cerevisiae are commonly used and fermentations carried are carried out in the temperature range 16-25° C. Yeast harvested at the end of the fermentation can be repitched up to seven times into subsequent fresh brews.

Yeast converts sugars present in wort to ethanol during fermentation. In the process, it also produces carbon dioxide, higher alcohols, organic acids, esters, aldehydes, ketones and sulphur compounds which affect the sensorial profile of beer. Different strains of yeast and wort compositions can yield characteristic flavours to a brew. However, in any form of microbial contamination (for example, bacteria, non-yeast fungi, wild yeast) of the brew or the vessels may lead to very negative and unpredictable palatability and may cause adverse health effects to the consumer. Microbial metabolism during fermentation and spoiling of food can result in histamine production, which is a major mediator in inflammation and allergy.

Beer is one of the most popular beverages in the world. Approximately, 1-2 billion hectoliters of beer is brewed annually worldwide. In a commercial setting a single brewing vessel may contain thousands of hectoliters (Guinness, Dublin, Ireland 5200 hl, Whitbread, UK 1200 hl, Heineken, NL 4800 hl). Each brew may last from 4 days to 14 days depending on the desired product. The brewing vessel and the brew itself are hygienically maintained to prevent unwanted microbial contamination. However, occasionally other microbes in particular certain classes of bacteria may contaminate the brew itself or the hardware and in some cases the initial contamination may not be detectible at the brewery but may persist during the laagering and storage phase. Such bacterial contaminations can lead to non-palatable and/or unhealthy beverages with very short shelf life and can be very costly for the industrial brewer to the extent that the brand reputation may be ruined.

Fortunately, beer and the brewing environment have several selective antimicrobial properties that prevent bacterial contamination. The anaerobic and hyperosmotic brewing conditions, low temperatures during manufacturing process, hop oils and hop bitter substances in the brew are unfriendly for bacterial growth. The end product of the brew is acidic (has an pH-value approximately 4.5), contains approximately 5% (v/v) of alcohol and has very little of any easily digestible sugars and amino acids. In addition to these intrinsic factors, many stages of the brewing process reduce the potential for contamination, such as mashing, wort boiling, pasteurization, filtration, aseptic packaging and cold storage. However, beers without, or with, reduced levels of one or more of these antimicrobial hurdles (for example, low-alcohol and/or unpasteurized beers), are at risk of contamination. Furthermore, there are over two-dozen bacterial species that can tolerate the inhospitable brewery conditions and some of these are notorious for their beer spoilage properties. The most common beer-spoiling bacteria species are Lactobacilli and Paediococcus. It is almost next to impossible to keep the entire brewing operation in a clinically sterile environment and bacterial contamination can occur at varying stages post mashing. Inclusion of prophylactic antibiotics in the brew is unacceptable to the consumer and could lead to environmental disaster with the selection of antibiotic resistant varieties of beer spoilage and other bacteria. It is the brewer's duty to ensure that these microorganisms do not multiply and usually checks are carried out at various stages during the process to determine the number of any putative beer spoilage bacteria. If the bacterial contamination is negligible, i.e., no detectable increase in the number of bacteria over a period of time, practically no metabolic products will be produced, and the beer will not be damaged. However, if significant contamination occurs, it is necessary to remove the metabolic products of the microorganisms through the use of active carbon filtration. Nonetheless, microorganisms can be forced through by pressure surges and still damage the packaged beer with their metabolic products. Beer spoilage by bacteria can result in product withdrawal or recall. Industry sources indicate that product withdrawal of 3 weeks stock due to Lactobacillus contamination from a standard size brewery have resulted in losses of up to 3 million Euros. Product recall can be extremely damaging commercially to brand name, resulting in immeasurable commercial losses.

Red wine is made from the must or pulp of red or black grapes that undergo fermentation together with the grape skins. White wine is made by fermenting juice, which is made by pressing crushed grapes to extract a juice, and the skins are removed and then play no further role. To start primary fermentation, a yeast is added to the must for red wine and the juice for white wine, and the yeast converts most of the sugars in the grape into ethanol and carbon dioxide. For red wine making, there is a secondary fermentation step in which bacterial fermentation converts malic acid to lactic acid, which decreases the acid in the wine and softens the taste of the wine. The most common preservative used in wine making is sulphur dioxide, which is produced by adding sodium or potassium metabisulfite to the system. Sulphur dioxide has two primary actions, the first being an anti-microbial agent and the second an anti-oxidant activity. It may be added prior to fermentation and immediately after alcoholic fermentation is complete. In the making of red wine, sulphur dioxide is used at high levels. Without the use of sulphur dioxide, wines can readily suffer bacterial spoilage no matter how rigorous the hygiene of the wine making process is.

Yeast is used in wine making, where it converts the sugars present in grape juice or must into ethanol. The yeast is normally already present on grape skins as the white powder, which is known as a bloom. Whilst fermentation can be achieved with this bloom, the results may be unpredictable and so now normally a pure yeast culture is added to the must or juice, which quickly dominates the fermentation and represses the wild yeasts. This gives a reliable and predictable fermentation. Most added wine yeasts are strains of Saccharomyces cerevisae and different strains have different physiological and fermentative properties.

Defensins form part of the human innate immune response. They are naturally occurring antimicrobial peptides (AMP) which are positively charged peptides that interact with negatively charged microbial membrane components causing disturbances in their structure leading to fluid imbalances, thereby killing their target. HBD-3 is expressed in the human oral cavity and parts of the alimentary canal and is part of the normal bacteria flora. Defensins help to mechanically kill many pathogenic bacteria that they come in contact with. They are very effective when the bacterial count is low, as they are not produced in large quantities.

### OBJECT OF THE INVENTION

It is an object of the present invention to provide products and methods for the prevention of bacterial contamination of the alcohol production or brewing process. A further object is to provide a number of strains of yeasts which have been engineered to produce an antimicrobial peptide which is a defensin. Another object is to provide strains of yeast capable of producing human β-defensin-3 (HBD-3). It is also an object to provide a method of producing alcohols in which the growth of spoilage microorganisms is inhibited and the use of preservatives is minimised. The invention aims at preventing, most if not all, common bacterial contaminants encountered during the fermentation process. The invention also aims not to add any additional production costs or affect the taste of the brew.

### SUMMARY OF THE INVENTION

According to the present invention there is provided a fermentation strain of yeast capable of expressing a defensin peptide.

The fermentation strain of yeast can be any yeast species, which is used in the production of ale, lager, beer, stout, wine, rum or spirits, or any other alcoholic beverage. These species may also be a species used in bioethanol production, and other industrial non-potable alcohol productions. The alcohol production may be ethanol production.

Suitable species include Saccharomyces cerevisiae which is the predominant species responsible for alcohol fermentation including beers and ales, Saccharomyces bayanus var bayanus, Caccharomyces bayanus var. uvarum (or simply Saccharomyces. uvarum) which is adapted to low temperature fermentation during wine and cider production and Saccharomyces paradoxus which has been isolated from Croatian wines, lager yeasts, originally referred to as Saccharomyces carlbergensis, which are now classified as Saccharomyces pastorianus; ale yeasts which are predominantly Saccharomyces cerevisiae, but also Saccharomyces bayanus strains which have also been identified from beer. Other suitable species may be hybrids between Saccharomyces cerevisiae and either Saccharomyces kudriavzevii, Saccharomyces bayanus var. uvarum, or Saccharomyces bayanus var. bayanus which have been isolated in wine fermentation and multi-hybrid strains of species within the Saccharomyces stricto sensu genus including those containing genetic information from the newly identified Saccharomyces eubayanus. The defensin peptide may be an alpha defensin, a beta defensin or a θ-defensin. The defensin may be a human defensin, or it may be derived from another species, either fungal, animal or plant. In particular the defensin may be the human β-defensin-3 (8BD-3). The defensin peptide may also be human P-defensin 1-6, or 26-28 or other human defensins. Also suitable are animal defensins selected from the group comprising from King Penguin, Ostrich, Rat, Mouse, bovine or insect defensin.

The yeast strain may carry the gene for the defensin on an episomal plasmid. Alternatively the gene may be integrated into the genome of the yeast. The gene may be inserted into the yeast genome downstream of a yeast alpha-factor secretory signal. The gene may be placed under the control of a yeast promoter. Suitable yeast promoters include but are not limited to GAL1, TEF, MAL11 and PGK or heterologous promoters such as the THD3 promoter from other yeast species such as Kluveromyces lactis.

The invention also provides a method of alcohol production comprising use of a yeast strain capable of expressing a gene for a defensin peptide. The method may further comprise a freezing step in which the fermentation mix is frozen and subsequently thawed.

In a still further aspect the invention provides a yeast biomass comprising a defensin peptide. This biomass may find use as a neutraceutical, or as a human or animal feedstuf or in the production of biogas.

The invention successfully produced sufficient quantities of HBD-3 in the fermentation through both methods and it has been shown, using a developed bioassay, that both in laboratory and micro-brewery setup, the expressed and secreted peptide can control and eliminate exogenously introduced beer spoilage micro-organisms. To date, no other party has described the use of defensins for the prevention of beer spoilage. It has been shown that HBD-3 is synthesized by actively growing yeast from an integrated copy of its gene driven by the constitutive promoter for the phosphoglycerate kinase (PGK) gene or from a heterologous promoter such the K. lactis Glyceraldehyde-3-phosphate dehydrogenase gene or episomally from the inducible galactokinase 1 (GAL1). The peptide can be secreted into the medium from the cells by incorporating either the alpha factor secretory signal or the invertase secretory signal sequence upstream of the defenisin gene sequence. Thus, during the initial expansion of the yeast culture and during the early part of the brewing phase, small but adequate amounts of HBD-3 is produced and secreted. A simple quick freezing of the brew improves the activity of HBD-3 against spoilage bacteria and reduces bacterial load by up to 90-95%. The peptide is most effective in fermentations carried out at about 20° C. to about 30° C. Thus HBD-3 adds another layer of protection against the beer spoilage microorganisms up until it is consumed. The presence of trace amounts of HBD-3 improves the storage of the yeast biomass for subsequent pitching. Furthermore, the yeast biomass is a valuable source of nutrients for the animal industry. There is a prophylactic regime of conventional antibiotic usage in many poultry and animal farms especially for pigs and other domesticated ungulates. The emergence of antibiotic resistant forms of pathogens has been linked to the wide-ranging use of antibiotics, prophylactic or otherwise. The use of HBD-3 containing yeast biomass as feed would achieve a similar prophylactic protection thus reducing the development of antibiotic resistance.

Antimicrobial assays demonstrate that the expressed gene reduces the bacterial load in fermentations. Antimicrobial activity is greatly enhanced by incorporating a freeze-thaw cycle into the process indicates that the HBD-3 activity increases the sensitivity of the bacteria to freezing.

Pilot industrial scale fermentations (50 L) indicate that the presence of the HBD-3 gene did not affect the beer quality. A beer spoilage challenge in the bottled product did not reveal any protective effects of HBD-3, although the test utilized larger than usual amounts of bacteria in the challenge.

The expression of small quantities of HBD-3 by the brewery yeast (lower than usually present in the human mouth and alimentary system) prevents bacterial contaminants in beer. Defensins present in beer are subsequently destroyed by acids present in the stomach and thus are harmless to humans. At the end of fermentations, the yeasts are removed by filtration and may be reused for subsequent pitching of fresh brews. The yeast biomass is often used as an important component of balanced animal feed. Yeast biomass is also used for human consumption as a rich source of micronutrients. The presence of any residual HBD-3 in the yeast biomass has the potential to provide protection from pathogenic bacteria to the end-user (animals or humans). This is particularly interesting in light of growing antibiotic resistance as a result of the liberal use of antibiotics.

The invention not only provides a prophylactic mechanism to prevent spoilage of yeast fermentations but additionally provides added neutraceutic value to the product as the small quantities of the antimicrobial peptide remaining in the lager can enhance the natural levels of β-defensin in the oral cavity. Defensins are important in maintaining the natural balance of the normal flora of the oral cavity and to protect against bacterial infections.

### BRIEF DESCRIPTION OF THE DRAWINGS

FIG. 1. pGREG506 plasmid. The location and sequence of the reel and 2 sites is shown. The restriction sites at the sites of homologous recombination are shown.

FIG. 2 Amino acid sequence of the HDB3 (Bold) containing the a-factor secretor) signal peptide (A) and the S. cerevisiae invertase secretory signal peptide (B).

FIG. 3. Strategy for integrating HBD-3 into the lager yeast genome.

FIG. 4. Strategy for removal of KanMx cassette from the integrated HBD-3 gene

FIG. 5 A. Minimum Inhibitory Concentrations (MIC) and B. LD-100 values of synthetic HBD-3 against gram positive and gram negative bacteria.

FIG. 6. Bactericidal effects of HBD-3 in beer fermentations.

FIG. 7. RT-PCR products of HβD-3 mRNA from CMBS-505V and CMBS-506V. Lanes 1, 2, & 3; CMBS-33, CMBS-505V and CMBS-506V grown in YEPD, respectively. Lanes 4, 6, 8 & 9 represent CMBS-505V grown in YEPGal. Lanes 5 & 7 represent CMBS-506V grown in YEPgal. Results obtained for lanes 1, 2, 3, 4, 5 & 8, reactions carried out in the presence of reverse transcriptase. Lanes 6, 7 & 9, reactions carried out in the absence of reverse transcriptase. Lanes 10 & 11; pGREG-505 plasmid and H2O respectively. Lanes 1-7, 10 & 11; RT-PCR/PCR carried out using HβD-3 primers, Lanes 8 & 9; RT-PCR carried out using actin primers.

FIG. 8. ELISA detection of HBD-3. The culture supernatants were concentrated 1000× and capture ELISA was performed using anti-HBD-3 antibody. HBD-3 concentration in the supernatants (boxed values) were extrapolated from the standard curve generated from known concentrations synthetic HBD-3.

FIG. 9 Reverse Phase HLPC trace of HUD-3, CMBS, CMBS-505 and CMBS-506 indicated by arrows.

FIG. 10. HBD-3 detection in RP-HPLC fractions.

FIG. 11. In situ activity of HBD-3 during fermentations at 11° C. Fermentations were seeded with 10² cfu/mL of Lb. brevis. Samples were taken from the above fermentations on Day 2 of seeding. Grey columns represent Lb. brevis viability directly from fermentations. Black columns represent Lb. brevis viability after freezing.

FIG. 12. Competitive Genomic Hybridisation Microarray Analysis of strains CM-PGK-51, CM-PGK-38, and CMBS. Differentially labeled genomic DNA were competitively hybridised to S. cerevisiae microarrays. ROH is shown on the Y-axis and the ORF location is shown on the x-axis. A region of chromosome XVI where the HBD3 gene was integrated is shown. The legend is shown to the right of the figure and arrows show the location of traces for each set of compared strains.

### DETAILED DESCRIPTION OF THE DRAWINGS

The human β-defensin-3 (HBD-3) was chosen as the suitable AMP molecule for the prevention of beer-spoilage by beer spoilage bacteria as it is well characterised and forms part of the natural bacterial flora of humans. Two approaches have been taken to express HBD-3 in the lager yeast strain CMBS. Firstly, the gene was expressed under the control of two different promoters (GAL1 and TEF1) on the episomal plasmid pGREG. Secondly, the HBD-3 gene has been inserted into a region of the yeast chromosome under the control of the constitutive promoter phosphoglycerate kinase (PGK) or the heterologous K. lactis TDH3 gene promoter. The site of integration on chromosome 16 at YPR159C-A was chosen because (i) we have prior knowledge of the genomic organization of the region and (ii) the nonessential nature of the region. Thus HBD-3 gene integration in this region does not result in inviability.

### Strategy

Identify one or more suitable AMPs for expression in yeast.

Reverse-translate the HBD-3 coding region using yeast codon bias and make a synthetic copy of the gene. Incorporate a secretory signal sequence upstream of the defensin gene so as to direct secretion of the peptide into the medium.

Use a low copy number expression plasmid to engineer the production of HBD-3 in yeast.

Introduce the plasmid-expressed HBD-3 into a brewery-yeast strain resistant to low concentrations of HBD-3.

Test the antimicrobial effects of the HBD-3.

Integrate the HBD-3 expression cassette into the brewery yeast genome and test the expression of HBD-3.

Test the antimicrobial effects of the HBD-3 produced in brewery yeast against beer spoilage microorganisms.

Examine the effect HBD-3 expression on the drinking quality of the beer.

### Materials and Methods

Brewery Yeast. The brewery yeast strain used was CMBS-33 (commercial bottom fermenting lager strain; Centre for Malting and Brewing Collection, Kasteelpark Arenberg 22, 3001 Leuven, Belgium) and was obtained from Professor Johan M. Thevelein, Department of Biology, K. U. Leuven and stocks maintained in our laboratory for the past 10 years. We have adapted this strain for high-gravity brewing as well as thermotolerance (James et al, 2008). Such adaptations have distinct effects on the genetic fingerprint of the strain. For genetic manipulations, this strain is grown in 2% maltose containing 1% Yeast Extract and 1% Bactopeptone (1×YEPM) supplemented with G418 antibiotic (200 μg/mL) where necessary. For brewing purposes pre-aerated wort at 1.060 g/mL density (15° Plato) with or without G418 antibiotic was used.

Laboratory Yeast. For various transient genetic manipulations and testing expression levels of HBD-3, we used the laboratory Saccharomyces cerevisiae yeast strain S1502B (MATa, his3-Δ1, leu2-3,112, trp1-289, ura3-52). This strain is grown in 2% glucose containing 1% each of Yeast Extract and Bactopeptone (1×YEPD) supplemented with G418 antibiotic where necessary. Alternatively, a 2% glucose or 2% galactose synthetic complete media lacking either histidine and/or uracil was used when auxotropic selection was required.

### Fementations.

Yeast strains were grown up from single colonies in 3 mL YEP-Maltose medium (YEPM) and expanded to 2 liters. 40% (w/v) wort was reconstituted in water from spray-dried malt (Belgium) supplemented with 1 mM Zinc and autoclaved. The wort was diluted to give 1.02-1.060 g/mL density (10-15° Plato), and aerated. The yeast harvested from the 2 liter YEPM medium was resuspended in the diluted wort to give a yeast concentration of 1−1.5×10⁷ cells/mL in either 250 ml sterile glass cylinders or in 2 L cylindro-conical EBC vessels. Wort was aerated once with compressed air, bubbling from the bottom of the vessel and anaerobic conditions were maintained by overlying with mineral oil. Flasks were incubated at a 45° angle, and incubated at 11-13° C. The density of the solution was measured every other day for 14 days. Samples were also used for the measurement of HBD-3 in the medium. At the end of the brewing period, yeast were recovered and stored for re-pitching if required. To test the efficacy of the HBD-3-yeast to prevent beer spoilage, individual beer spoilage bacterial strains (10³/mL) were introduced along with pitching yeast. The surviving bacteria were quantified every other day along with viable yeast cells in the brew. The physical and chemical characteristics of the brew as well as taste and palatability were also measured with the help of healthy volunteers. This test was repeated with a synthetic complete media with maltose as the carbon source to rule out any bacteriocidal effect of the brew components.

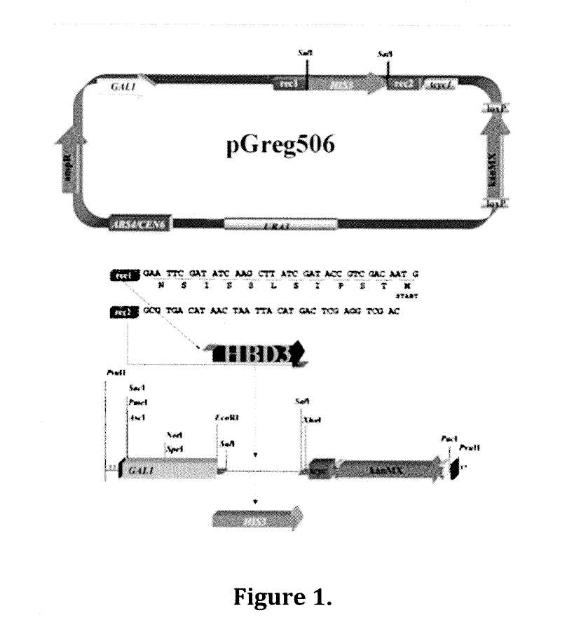

Plasmids. In the course of the experimentation, we have used three yeast-bacteria shuttle vectors (pGreg505, 506 and 599) and a bacterial vector (p-Bluescript SK+). The bacterial vector was used to synthetically assemble the yeast secretion signal, HBD-3 coding and termination sequences (HBD-3 cassette) optimized for translation in yeast. pGreg series of vectors were used to test the expression of the HBD-3 in both laboratory and brewery yeast strains. pGreg vectors can be used for cloning by homologous recombination in yeast (FIG. 1). pGreg505 has auxotrophic markers for histidine and leucine. It also has KanMx gene for selection on G418 substrate. A coding frame of interest, such as the HBD-3 cassette, replaces the histidine marker. Thus following, homologous recombination, the transformants are unable to grow in the absence of histidine. Plasmids pGreg506 contains an alcohol dehydrogenase gene (ADH) terminator sequence followed by the cytochrome C1 (CYC 1) termination sequence, while pGREG505 contains CYC1 gene terminator sequence only. pGreg506 contains a URA gene while pGreg505 contains a LEU gene. pGREG vectors are described in Jansen, Wu et al 2005.

### Design of HBD-3 Cassette.

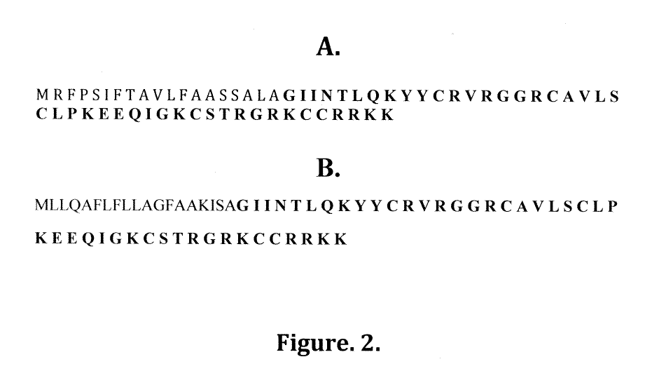

The human β-defensin-3 (HBD-3) amino acid sequence (46 amino acids) was obtained from the Protein Data Base (PDB). A web based interface was used to ‘reverse translate’ this sequence into the corresponding nucleotide sequence using the codon usage for yeast. A complete synthetic version of the gene was synthesized. The DNA sequence for the Saccharomyces cerevisiae α-factor secretory signal peptide (19 amino acids) was incorporated at the 5′ end of the DBD-3 sequence in-frame (FIG. 2A). Alternatively, the signal sequence from the S. cerevisiae Invertase gene was used (FIG. 2B). The α-factor signal sequence-HDB3 synthetic gene was cloned into pBluescript at the EcoR1 site.

The Rec1 Forward primer contains 30 nucleotides upstream of the Sal I restriction site of the pGreg506 plasmid followed by 30 nucleotides starting with the ATG of the signal sequence-HBD-3 open reading frame (FIG. 1). The Rec2 Reverse primer (FIG. 1) likewise has at the 3′ end, the reverse complement of 30 nucleotides upstream of the translation termination codon followed by 30 nucleotides downstream of the Rec2 site. The Rec2 site is followed by a short stretch of mRNA termination sequences from the CYC gene in pGreg plasmid series. The KanMx gene cassette is downstream of the termination sequences and is flanked by Loxp direct repeat sequences (FIG. 1). The Rec1 and Rec2 primers were used to generate a PCR product using the signal sequenceHDB3 synthetic gene pBSK plasmid as a template. The resultant PCR product contains the signal sequence-HBD-3 gene flanked by the Rec sites. The PCR product was incubated with both pGreg505 and pGreg506 vectors digested with SalI and the mixture was introduced into the S150-2B S. cerevisiae strain of yeast. Digestion with SalI cuts out His gene fragment providing the entry sites for recombination. Recombination between the pGreg506/505 plasmids and the PCR fragment results in hybrid plasmids which are Ura⁺, G418 resistant, His⁻ or Leu⁻, G418 resistant and His⁻. The resultant plasmid places the HBD-3 gene under the control of the GAL promoter. The recombinants were identified through yeast colony PCR using primers located in HBD3 and the plasmid. The positive plasmids (herein referred to as GAL-HBD-3-505V and GAL-HBD-3-506V) were rescued from yeast and transformed into E. coli bacteria and plasmid DNA isolated from E. coli was sequenced to ensure the integrity of the insert. The plasmids were also transformed into the lager yeast strain CMBS-33 and transformants were selected as G418 resistant. Aliquots of all transformants were cryopreserved at −70° C.

### Chromosomal Integration of the HBD-3 Cassette in S150-2B and CMBS-33.

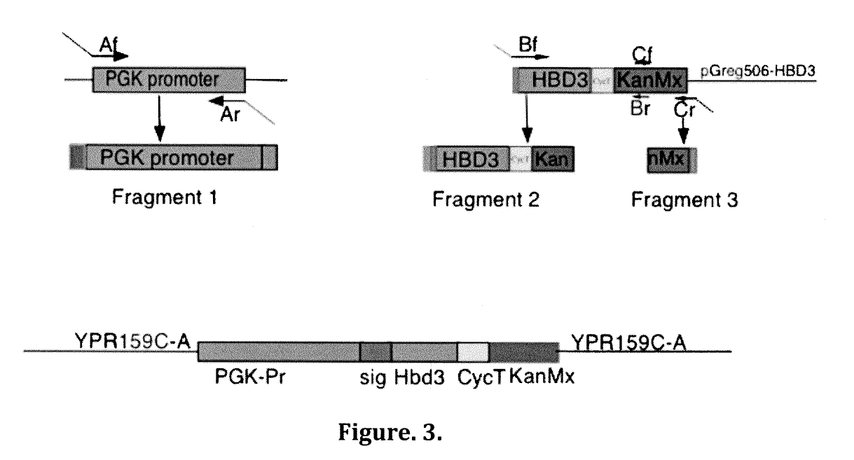

The HBD-3 cassette was integrated into the brewery yeast CMBS at the YPR159C-A location on the right arm of the chromosome XVI using standard procedures for integration known to artisans in the field. The location was chosen because of our knowledge of the copy number and gene sequence of the region and the fact that the region contains genes that are either non-essential or redundant. As part of the integration, the inducible Gal-1 promoter was replaced with the phosphoglycertate kinase (PGK) promoter. Briefly, DNA sequences corresponding to the S. bayanus PGK promoter were amplified using primers Af and Ar (FIG. 3). The 5′ region of primer Af contains sequences complementary to DNA sequences at the YPR159C-A and the 3′ end has sequences complementary to the PGK promoter. Likewise, the 5′ end of primer Ar has homology to the start ATG on the signal peptide and 30 nucleotides downstream. The 3′ end is complementary to the 3′ end of the PGK promoter. Primers Af and Ar amplify fragment 1 (FIG. 3). Primer Bf (FIG. 3) primes from the ATG of the signal sequence and has a 5′ region of 30 nucleotides representing the 3′ end of the PGK promoter. Primer Br (FIG. 3) is located in the middle of the KanMx gene. Primers Bf and Br together amplify fragment 2, which has all of the HBD-3 coding and termination sequences and part of the KanMx gene.

Primers Cf and Cr (FIG. 3) together amplify fragment 3, the reminder of the KanMx cassette that has extensive overlap with fragment 2 at its 3′ end. Primer Cr contains 30 nucleotides at the 5′ homologous to the YPR159C-A, downstream of the sequences present in primer Af. The three fragments were amplified separately and purified. Equimolar amounts were used in a transformation reaction with CMBS-33 using standard procedures with the following exceptions; the PEG-lithium acetate incubation was at 30° C. for 16 hours and the recovery period was extended to 24 hours. The cells were plated on to YEPM-agar plates containing 200 μg/mL of the antibiotic G418 (YEPM-G418) and incubated up to 72 hours. Colonies were picked on to a YEPMG418 master plate. To indentify integrants to the YPR159A site, colony PCRs were carried out using primers anchored in the genomic DNA and various fragment specific primers.

Removal of the Marker Sequences from the Integrated Copy of the HBD-3 Gene Cassette.

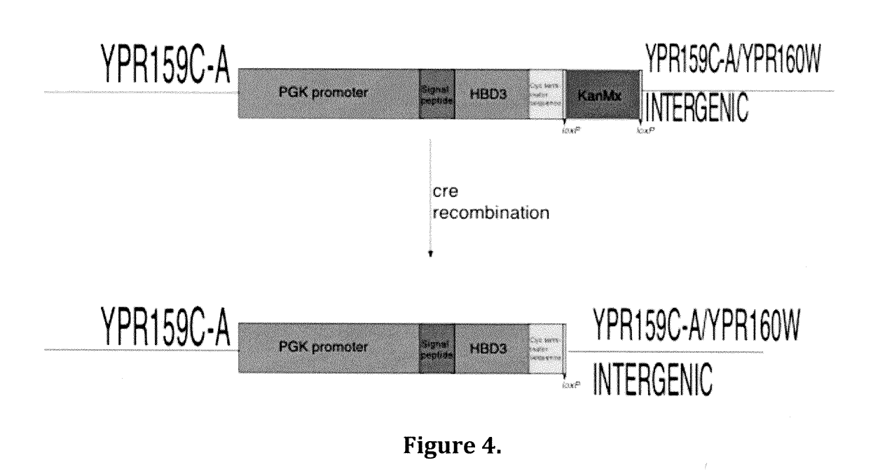

The integrated copy of HBD-3 contains has the KanMx gene conferring resistance to G418 amyloglycoside. This phenotype was necessary to select for the transformants on G418 containing YEPM-agar plates. The KanMx cassette is flanked by 34 base pair loxP direct sequences (FIG. 4). The core of the direct repeat sequences are recognized by the recombinase enzyme cre, which cleaves them at their core, and recombines the ends to recreate one of the loxP sites, loosing the sequences in between. In order to induce the recombination in the yeast integrated copy of loxP, cre enzyme must be supplied in vivo. A plasmid bearing a galactose inducible copy of the cre gene was introduced into yeast strains bearing the integrated cassette and was selected for using the drug resistant marker gene phleomycin. The phleomycin resistant yeast colonies were grown up in YEP-raffinose (YEP-R) medium to optical density 0.6, harvested, washed and transferred to YEP-galactose (YEP-Gal) medium for 2 hours. The cells were recovered and plated on YEP-maltose (YEPM) plates. The resulting colonies were triplica plated onto YEPM and YEPM-phleomycin and YEPM-G418 plates. Colonies that are sensitive to G418 but resistant to phleomycin were grown up without selection for 12 hours and this step was repeated for a further 24 hours each time diluting 1:1000. An aliquot of the final culture was plated first on to YEPM plates and the resulting colonies were triplica plated on YEPM, YEPM-phleomycin and YEPM-G418 plates. The colonies that grew up only on YEPM were tested by PCR for the integrity of the PGKHBD-3 gene cassette. Colonies that were G418 and phleomycin sensitive but that contained PGK-HBD-3 were selected.

### Promoter and Signal Sequence Swaps.

The PGK promoter was replaced with a TDH3 promoter from K. lactis strain NRRL Y-1140 (chromosome A: co-ordinates 1025294-1025790:496 nucleotides, Genebank Accession Number [R]82121) by homologous recombination as described above. Likewise the α-factor secretory signal was replaced with the invertase secretory signal (FIG. 2B). This generated strains CM-KLTHD3-51 and CM-PGK-Inv-51 respective.

### Detection of Expression of Recombinant HBD-3.

Cultures of the S. cerevisiae yeast strain S150-2B or the S. pastorianus strain CMBS, transformed with GAL-HBD-3-505V and GAL-HBD-3-506V or the parental plasmids pGreg506/505 were grown in YEPD. They were expanded to 100 mL cultures in YEP containing 2% raffinose to an optical density of 0.6, at which time the cells were harvested, washed twice in sterile water. The pellets were resuspended in YEP-containing 2% galactose at a concentration of 0.2 OD and grown for a period of 4-6 hours. The CMBS strain PGK-HBD-3int was grown in YEPM. Reverse Transcriptase analysis of HBD-3 RNA levels were carried out as previously described (Canavan and Bond, 2007).

The cell culture supernatants were collected and assayed for the presence of HBD-3 using ‘antigen capture’ ELISA (Peprotech). Briefly, 100 μL of affinity purified polyclonal anti-HBD-3 antibodies (3 μg/mL) in PBS were immobilized on Maxisorb Nunc immunoplates. Following washing and blocking of the wells with 1% BSA in PBS, 100 μL cell culture supernatants were added. Following a 2 hour incubation, the wells were emptied and washed 4-times in PBS-Tween 0.05% and incubated with horse-radish-peroxidase (HRP) coupled Anti-DBD-3. The bound antibody was identified through colorimetric reaction with 3,3′5,5-Tetramethylbenzidine (TMB). Colour changes were measured at 620 nm and in most cases enhanced by the addition of H2SO4 to 0.2 M and the measurement repeated at 420 nm. A standard curve using synthetic HBD-3 was included in each ELISA.

### RP-HPLC Analysis of Secreted HBD-3

Supernatants from yeast cells grown in SC-Gal were concentrated 10-fold, reconstituted in buffer A (0.1% TF/\) and applied to a C18 column reverse-phase HPLC column (Zorbax 300SB-C18-Agilent). Proteins/peptides bound to the column were eluted using a concentration gradient of acetonitrile (0-60% in Buffer A). Fractions were collected at a rate of 1 mL/min. The sample was then lyophilized and resuspended in 120 μl of SDW.

### Functional Bioassay to Detect Recombinant HBD-3.

Cultures of Lactobacillus brevis L1055 or a control bacterium Staphylococcus aureus-were cultured at 37° C. overnight in MRS media (10 g litre⁻¹ universal peptone, 5 g litre⁻¹ meat extract, 5 g litre⁻¹ yeast extract, 20 g litre⁻¹ D(+)-glucose, 2 g litre⁻¹ K2HPO4, 2 g litre⁻¹ diammonium hydrogen citrate, 5 g litre NaOAc, 0.1 g litre⁻¹ MgSO4, 0.05 g litre⁻¹ MnSO4, final pH 6.5+/−0.2 at 37° C.). To prevent yeast growth in samples taken from fermentations seeded with L. brevis, the MRS medium was supplemented with cycloheximide at 10 μg/ml. The cultures were diluted to give a final densitiy of approximately 10³ cells/mL. Synthetic HBD (0.5-10 μg/mL) was added to the bacterial cultures in a final volume of 100 μl volumes. CMBS cultures containing the pGREG, HBD-3-505V or HBD-3-506V were grown in overnight synthetic medium plus galactose (2%). The supernatants were concentrated (1000-fold) using molecular filtration membranes [Molecular Weight Cut Off (MWCO) 2000] ensuring all unspent sugar was removed from the medium. The concentrated supernatants were added to the bacterial cultures as described above. Following a two-hour incubation at 37° C., the number of bacterial colonies in each sample was determined following incubation on MRS plates containing cycloheximide at 37° C. for 48 hrs.

### Results:

### Beer-Spoiling Bacteria are Sensitive to HBD-3 In Vitro and Under Fermentation Conditions.

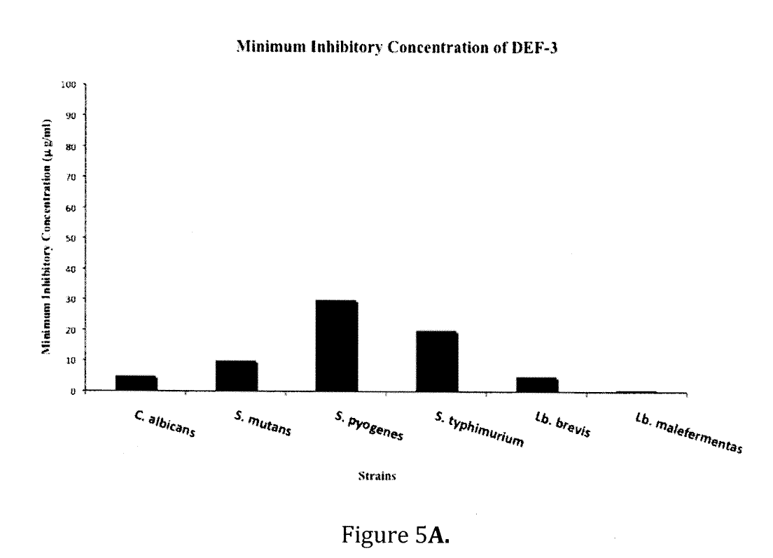

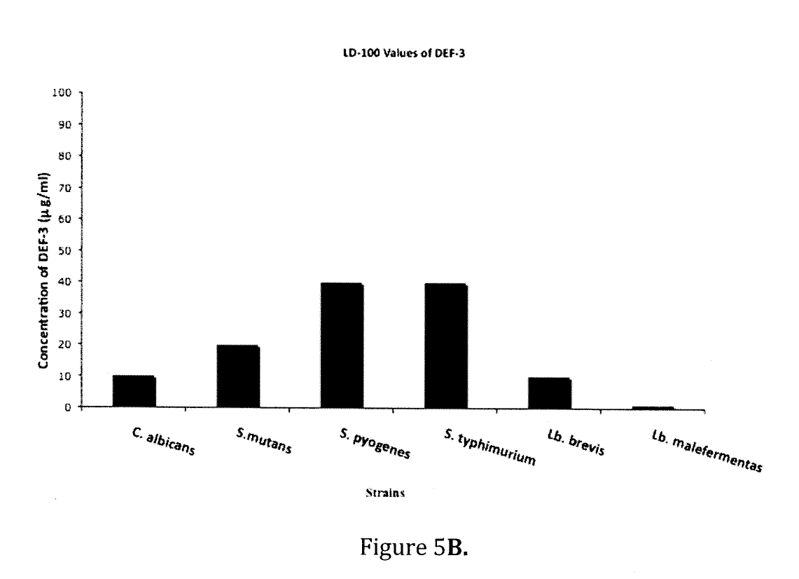

HBD-3 has previously been shown to exert a strong antimicrobial activity against both gram-positive and gram-negative bacteria, although its activity against the gram-positive Lactobacilli and Paediococci species, most commonly found as beer-spoiling bacteria, has not been thoroughly examined. To test the activity of β-Defensin-3 against beer-spoiling bacteria, a defensin bioassay was carried out as described in the Methods section. Since our goal was to express HBD-3 in lager yeast, the cytotoxic effects of HBD-3 against the lager yeast CBMS was also examined. As shown in FIGS. 5A and B, Lactobacillus brevis and Lactobacillus malifermentus were effectively killed at concentrations of 10 and 1 μg/mL respectively. The minimum inhibitory concentration (MIC) values for HBD-3 against Lb. brevis and Lb. malefermentas were 5 μg/ml 0.5 μg/ml respectively. The LD-100 and MIC were lower than that required against other gram positive strains such as S. mutans, S. pyogenes, and S. typhimurium. The lager yeasts were resistant to HBD-3 at concentrations of up to 100 μg/ml, indicating that it should be possible to express HBD-3 in lager strains without reducing the viability of the yeast. (data not shown).

### Detection of HBD-3 Activity in Beer Fermentations

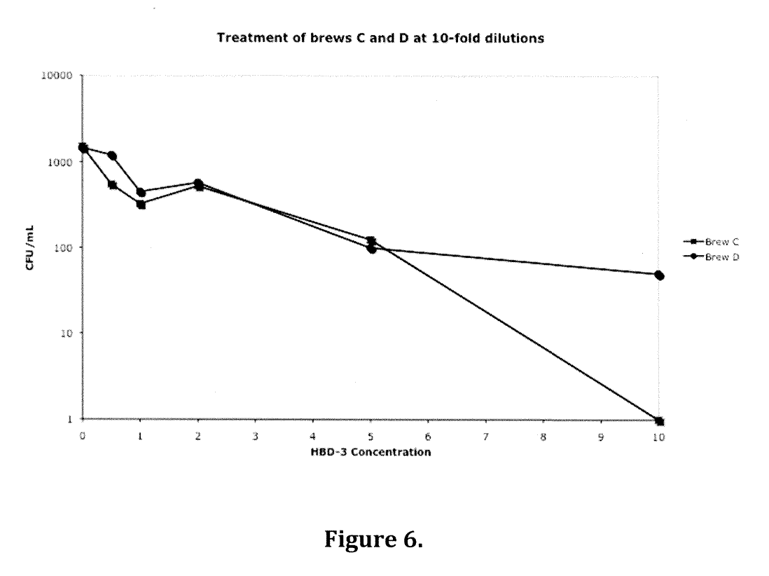

To determine if HBD-3 is effective against BSMs under fermentation conditions, CMBS strain was fermented under standard conditions and seeded on Day 6, with Lactobacillus brevis 1105 at 10², and 10⁴ cfu/ml. Fermentations were continued for a further 8 days. On day 14, samples of the brew were incubated with increasing concentrations of HDB3 as described in the Methods section. Surviving bacteria were enumerated by plating on MRS medium, supplemented with cycloheximide. As shown in FIG. 6, the bacterial load in the wort was reduced 100-fold at HBD-3 concentrations of 5 μg/mL in two independent fermentations, thus indicating the HBD-3 is effective against Lactobacilli under fermentation conditions.

Expression of HBD-3 on pGREG Plasmid in CMBS.

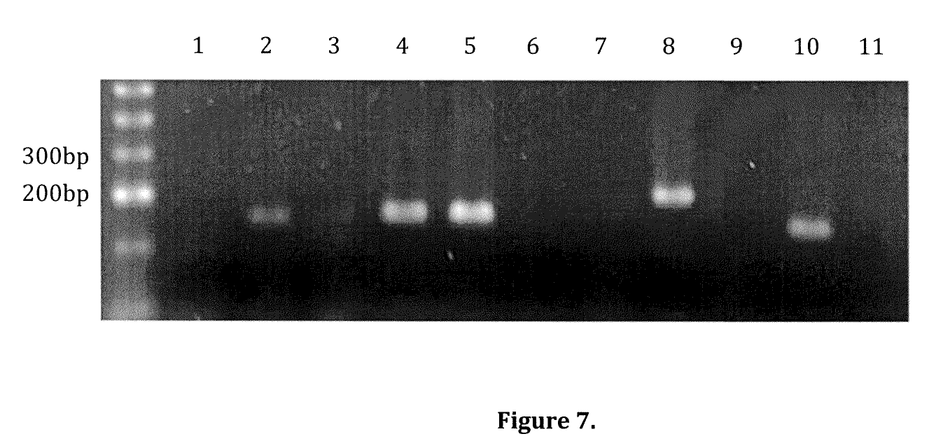

Having demonstrated the HBD-3 is effective against L. brevis under fermentation conditions, we next set out to express HBD-3 in the lager strain CMBS. The HBD-3 gene, contiguous with the DNA sequence encoding a modified S. cerevisiae α-factor signal sequence, was commercially synthesized as described in the Methods section and inserted into the pGreg vectors 505 and 506 using in vivo homologous recombination under the control of a GAL1 promoter. Reverse transcriptase-PCR analysis of RNA extracted from cells grown in glucose or galactose demonstrated that the gene was efficiently transcribed under the inducible conditions (FIG. 7).

### Detection and Characterisation of HBD-3 in the Supernatants of Brews.

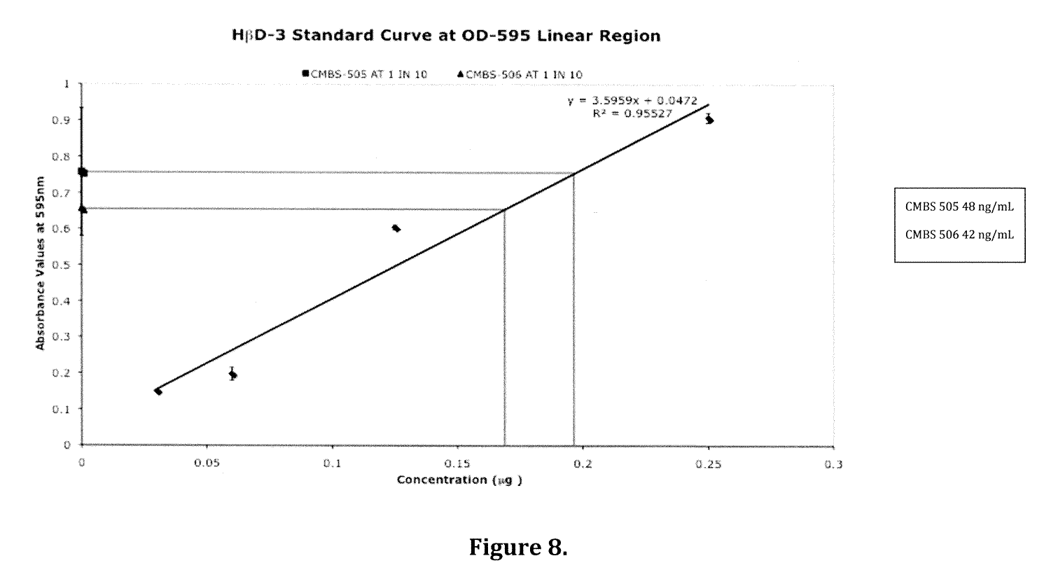

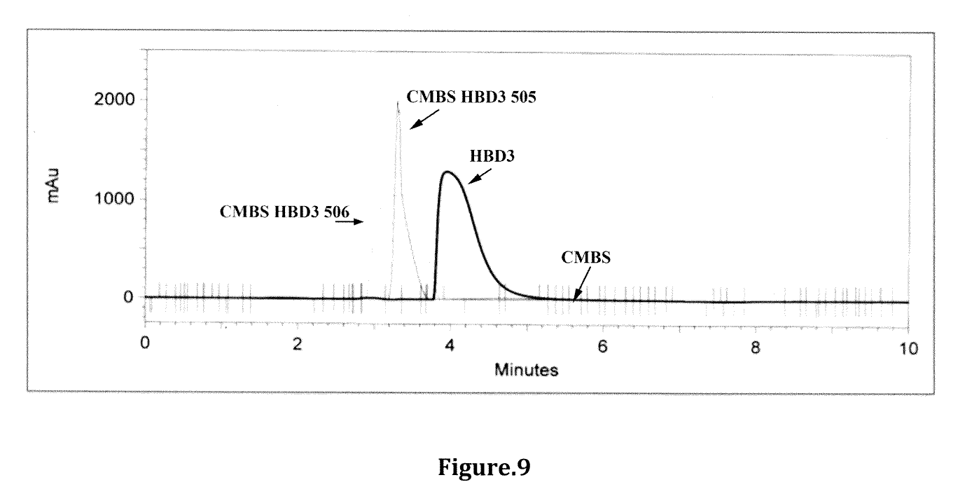

The expression of functional HDB3 was examined by capture ELISA using an antiHBD-3 antibody. Cells were grown in YEPGal and the supernatants were concentrated 1000×. Approximately 40-50 ng/mL of HBD-3 was detected in the supernatants (FIG. 8). The secreted HBD-3 was further characterized by Reverse-Phase High Performance Liquid Fractionation (RP-HPLC). Yeast cells expressing HBD-3-505 and HBD-3-506 were grown in minimal medium containing 2% galactose. The supernatants were then concentrated 10-fold and applied to a C18 column reverse-phase HPLC column. Peptides bound to the column were eluted using a concentration gradient of acetonitrile (0-60%), lyophilized and resuspended in water. As shown in FIG. 9, a major peak was observed for the synthetic peptide in fractions 4 and 5 (3-4, and 4-5 minutes respectively). Supernatants from both HBD-3-505 and HBD-3-506 produced a sharp peak in fraction 4 (3-4 minutes), slightly ahead of the synthetic HBD-3 peak. No peak was observed with supernatants from CMBS lacking the plasmids. The slight difference in elution profiles between the synthetic peptide and the expressed peptides may reflect post-translational modification of the peptide in the yeast strains and/or alterations in the sulphydyral bonds between the many cysteine residues in the peptides.

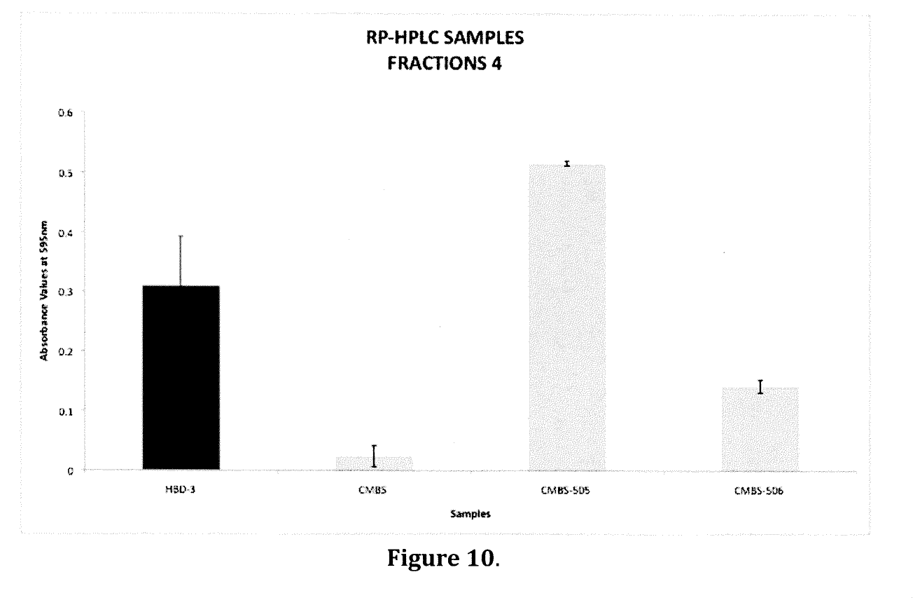

To determine if the peptide peaks represent HBD-3, ELISA assays using an anti-HBD-3 antibody were carried out. As shown in FIG. 10, HBD-3 was detected in the fraction containing the major peak (fraction 4, 3-4 minutes). No HBD-3 activity was detected in supernatants from CMBS cells lacking the plasmid.

### Biological Activity of Expressed HBD-3 Against Beer-Spoiling Bacteria Under Fermentation Conditions.

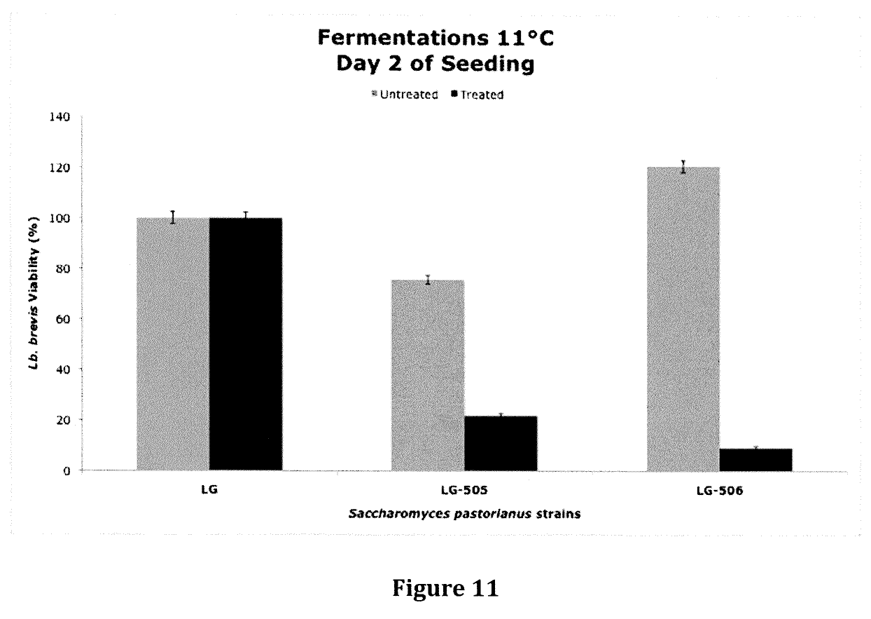

Experiments were carried out to analyse the in situ antimicrobial activity of HBD-3 secreted from yeast strains transformed with CMBS-505V and CMBS-506V, under fermentative conditions. Wort (16° P) was pitched with 1.5×10⁷ cfu/mL of yeast cells. Fermentations were carried out at room temperature (RT) and 11° C. (FIG. 11). The fermentative performances of both CMBS-505 and -506 were comparable to the parental strain CMBS. All three strains produced approximately 3.6% alcohol after 10 days, indicating that the presence of the vector did not interfere with fermentation (data not shown).

On the second day of fermentation, gram positive bacteria Lb. brevis were seeded at 10² cfu/mL (Day 0 of seeding). Bacterial viability was examined after a further 2 days of growth in the fermentations. As shown in FIG. 11, the bacteria count was reduced by 20% in the CMBS-505 fermentation compared to CMBS and CMBS-506, indicating some bactericidal activity. Following freeze thawing, bacteria counts were reduced by 80% in CMBS-505 and by 90% in CMBS-506. No reduction in bacterial count was observed in CMBS strains lacking the 505 plasmid. Similar results were obtained when cultures were seeded with a gram negative beer spoiling bacteria Pectinatus frisingensis.

Integration of HDB3 into the Genome of CMBS and Molecular Characterization of The Resultant Clones.

The data described above indicates that HBD-3 can be expressed in lager yeast strains, and secreted into the medium. The secreted peptide is effective at reducing the bacterial load of a brew, and the bactericidal effect can be enhanced by a short freeze-treatment of the brew. While effective at expressing HBD-3, plasmid constructs are inherently unstable and can be lost from the cell unless antibiotic selection is maintained. This is not sustainable or practical under industrial fermentation conditions. Therefore a copy of the HBD-3 gene was introduced into the genome of CMBS on chromosome XVI under the control of a Saccharomyces bayanus PGK promoter as described in the Methods section. KanMx-resistant colonies were selected and the presence and orientation of the integrated HBD-3 gene was confirmed by DNA sequence analysis (data not shown). The KanMx cassette was subsequently removed by the action of cre, which was introduced into the cells on a plasmid containing a pleomycin-resistance gene. The pleomycin plasmid was subsequently removed from the cells by growth and serial dilution of the cells.

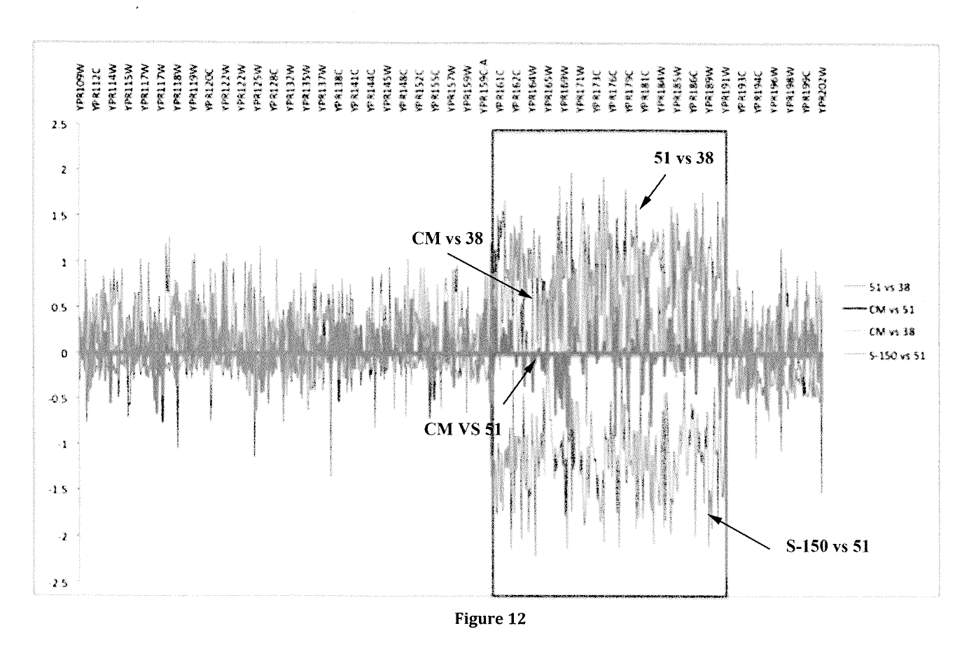

Two transformants that were G418^{s}, Pleomycin^{s} (CM-PGK-51 and CM-PGK-38) was further characterised by competitive genomic DNA hybridization (CGH) as previously described (Bond, 2004, James et al, 2008). Briefly, genomic DNA is extracted from each strain and labeled differentially with either Cy3 or Cy5. The two DNA samples to be compared are mixed together and hybridized to a microarray chip containing the entire S. cerevisiae genome arrayed in 25 nts sections. The differentially labelled DNAs compete for binding for the respective homologous region on the microarray chip. Regions where amplifications have occurred are depicted by increased ratio of hybridization (ROH) and regions of deletions are depicted by a decrease in the ratio of hybridisation. As shown in FIG. 12 CM-PGK-51 and the parent CMBS-33 have very similar gross genome organization, as indicated by log 2 ROH of close to 0. All the known copy number variations (CNV) of segments of the chromosomes are preserved in both the parent and the integrant CM-PGK-51. However, CGH analysis of the integrant CM-PGK-38 showed significant differences in the CNV of known segments of various chromosomes. In particular, the copy number of the YPR159C-A to YPR190 region showed a 3-fold increase. This region has previously been identified as being susceptible to chromosome rearrangement (Bond et al, 2004).

Analysis of the fermentation profiles of the integrated strains, revealed that CM-PGK-51 displayed a profile similar to the parent CMBS while CM-PGK-38 showed poor fermentation ability (data not shown). Therefore CM-PGK-51 was deemed the most suitable integrant.

Two further strains were developed. Firstly, the PGK promoter was replaced with the K. lactis TDH3 promoter to generate CM-KLTDH-51 and secondly, the α-factor secretory signal was replaced with an invertase secretory signal (CM-PGK-Inv-51). ELISA analysis of medium from cultures of grown in YPD for 24 hrs indicated that HBD-3 was secreted into the media (Table 1).

TABLE 1
HBD3 Levels in YPD after 24 hrs.

| Strain | ng/mL HBD-3 | St Dev |
|---|---|---|
| CM-PGK-51 | 13.84 | 2.5 |
| CM-PGK-38 | 8.76 | 0.42 |
| CM-KLTDH-51 | 12.84 | 3.6 |
| CM-PGK-Inv-51 | 14.80 | 3.2 |

Bioassays as described for yeast strains transformed with CMBS-505V and CMBS-506V in FIG. 11 above were also carried out with yeast strain CM-PGK-51. We observed a similar killing effect on both gram positive (L. brevis) and gram negative (P. frisingensis) when seeded into fermentations of the CM-PGK-51 strain. When seeded at a higher CFU of 1×10³ cells/mL, into fermentations carried out at 11° C., up to 59% cell killing was achieved after a freeze treatment. The killing effect was greatly increased when the fermentations were carried out at 20° C. At this temperature up to 95% killing was achieved.

Pilot scale fermentations (50 L) were carried out using CM-PGK-51 as a starter culture. Using a number of ECB standardised assays, the results indicated that the presence of the HBD-3 gene did not affect the beer quality (data not shown). A beer spoilage challenge in the bottled product did not reveal any protective effects of HBD-3, however a freeze-thawing cycle was not conducted as part of this experiment.

### CONCLUSION

Human β-defensin is effective in eliminating beer-spoiling bacteria from lager brews. A human β-defensin (HBD3) gene has been successfully cloned into an industrial beer yeast strain, episomally and as an integrated copy into the genome.

The expressed gene produces functional β-defensin that is secreted from the cell into the medium.

β-defensin is present in the spent medium at concentrations between 40-50 ng/mL.

β-defensin expressed in lager yeasts is effective in reducing the bacterial load of fermentations seeded with L. brevis and other beer-spoiling bacteria.

Introducing a freeze-thaw cycle into the process can increase the effectiveness of β-defensin.

Expression of β-defensin does not alter the beer quality, flavours and alcohol content. The invention will help reduce bacterial contamination in industrial fermentations.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A recombinant fermentation strain of yeast capable of expressing a gene encoding a defensin peptide.

2. A strain as claimed in claim 1 wherein the defensin peptide is an alpha defensin, a beta defensin or a θ defensin.

3. A strain as claimed in claim 1 wherein the defensin is a plant or animal defensin.

4. A strain as claimed in claim 1 wherein the defensin peptide is a human defensin.

5. A strain as claimed in claim 1 wherein the defensin peptide is selected from the group comprising the human β-defensin-3 (HBD-3), Defensins 1-6, and 26-28.

6. A strain as claimed in claim 3 wherein the animal defensin is selected from the group selected from King Penguin, Ostrich, Rat, Mouse, bovine or insect defensin.

7. A strain as claimed in claim 1 wherein the yeast is selected from the group comprising yeasts used in the production of ale, lager, beer, stout, wine, rum, spirits or other alcoholic beverages, bioethanol production, biogas production, or other industrial non-potable ethanol production.

8. A strain as claimed in claim 1 selected from the group comprising S. cerevisiae, S. bayanus var. uvarum, S. paradoxus, S. pastorianus (formerly S. carlbergensi) and hybrid yeast species used in industrial processes, particurlary hybrids between S. cerevisiae and either S. kudriavzevii, S. bayanus var. uvarum, S. bayanus var. bayanus, S. eubayanus or multi-hybrids of the above named species.

9. A strain as claimed in claim 1 wherein the gene encoding the defensin peptide is carried on a plasmid.

10. A strain as claimed in claim 1 wherein the gene encoding the defensin peptide is integrated into the genome of the yeast.

11. A strain as claimed in claim 6 wherein the gene is inserted into the yeast genome downstream of a yeast alpha-factor secretory signal.

12. A strain as claimed in claim 11 wherein the gene is placed under the control of a yeast promoter.

13. A strain as claimed in claim 11 wherein the promoter is selected from the group comprising GAL1, TEF, MAL11, PGK and THD3.

14. A method of alcohol production comprising use of a yeast capable of expressing a gene encoding a defensin peptide.

15. A method as claimed in claim 14 further comprising a freeze-thaw step in which the fermentation brew is frozen during production and subsequently thawed.

16. Alcohol whenever produced by a method as claimed in claim 14 or 15.

17. An alcohol as claimed in claim 15 which is beer, ale, lager, stout, wine, rum or other spirit, bioethanol, biogas or non-potable alcohol.

18. An alcohol as claimed in claim 16 with neutraceutical properties.

19. A yeast biomass produced as an end-product of the method of claim 14 or 15, comprising a defensin peptide.

20. A biomass as claimed in claim 19 for use as a neutraceutical or a human or animal feedstuff, or in the production of biogas.


SEQUENCE LISTING (4 sequences)

SEQ ID NO: 1 - DNA, 37 bp, Artificial Sequence
  Rec1 forward primer
gaattcgatatcaagcttatcgataccgtcgacaatg

SEQ ID NO: 2 - DNA, 35 bp, Artificial Sequence
  Rec2 reverse primer
gcgtgacataactaattacatgactcgacgtcgac

SEQ ID NO: 3 - protein, 64 aa, Artificial Sequence
  5′ end of HBD-3 sequence
MRFPSIFTAVLFAASSALAGIINTLQKYYCRVRGGRCAVLSCLPKEEQIGKCSTRGRKCCRRKK

SEQ ID NO: 4 - protein, 64 aa, Artificial Sequence
  Signal sequence from S. cerevisiae invertase
MLLQAFLFLLAGFAAKISAGIINTLQKYYCRVRGGRCAVLSCLPKEEQIGKCSTRGRKCCRRKK